(19) United States
(12) Patent Application Publication
(10) Pub. No.: US 2006/0293384 A1
(43) Pub. Date: Dec. 28, 2006

(54) Isethionyl nitrates and compounds thereof

(76) Inventor: Christopher J. Whewell, Georgetown, TX (US)

Correspondence Address: Christopher J. Whewell;Western Patent Group, 6020 Tonkowa Trail, Georgetown, TX, 78628, US

(21) Appl. No.: 10/993,469

(22) Filed: Nov. 19, 2004

Related U.S. Application Data
(60) Provisional application No. 60/619,633, filed on Oct. 16, 2004.

(51) Int. Cl. A61K 31/21; C07C 309/06

(52) U.S. Cl. 514/509; 558/483

(57) ABSTRACT

The present invention provides nitric ester compound corresponding to the formula: 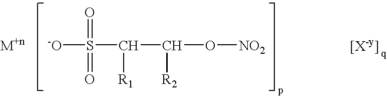
in which the cation M is selected from the group consisting of: hydrogen, alkali metal cations, alkaline earth metal cations, Group III metal cations, transition metal cations, ammonium ions (including organic-substituted ammonium cations), including mixtures of any two or more of the foregoing; R₁ and R₂ are each independently selected from the group consisting of: hydrogen and a C₁ to C₆ hydrocarbyl group; n represents the valence of the cation, including the integers 1, 2, and 3; X is an anion selected from the group consisting of: fluoride, chloride, bromide, iodide, nitrate, nitrite, phosphate, phosphate, monohydrogen phosphate, taurate, dihydrogen phosphate, monohydrogen sulfate, sulfate, carbonate, bicarbonate, hydroxide, carboxylate, dicarboxylate and polycarboxylate anions; y is the valence of the anionic species X and may be 1, 2, or 3; p may be any integer including 1, 2, and 3, subject to the proviso that p is not greater than n; q may be any integer selected from the group consisting of: 0, 1, 2, 3 subject to the proviso that q is not greater than n−p, and further subject to the provisos that the sum of p plus the product of (y times q) equals the valence n of the cation M and that p is not equal to zero. The compounds of the invention are useful as coronary vasodilators.

### CROSS-REFERENCE TO RELATED APPLICATIONS

This application claims the benefit of U.S. Provisional Application Ser. No. 60/619,633, filed Oct. 16, 2004, which application is incorporated herein by reference in its entirety.

### TECHNICAL FIELD

The present invention relates to chemical materials within the class of compounds known as nitric esters. More particularly, it relates to organic nitric esters having, in a preferred form of the invention, from about 2 to 4 carbon atoms, whose molecular structure also includes a sulfonate or sulfate group, and salts thereof.

### BACKGROUND INFORMATION

Nitric esters have been materials of interest for chemists since the time of their initial discovery, including products produced by Ascanio Sobrero from the nitration of sugars and polyhydroxy compounds, including the works of Alfred Nobel towards stabilizing such compounds.

It is well known that various nitric esters of sugars and poly-ol compounds have found widespread employment as an active constituent in explosive compositions of matter, of which Nobel's Safety Powder, blasting gelatin, and straight-dynamite are exemplary. These materials provided a great benefit to civil engineers and made many works possible, with one of the earliest examples being the construction of the Hoosac tunnel in the early state of Massachusetts.

It is also known that certain organic nitric esters provide a vasodilatory effect on the circulatory system of mammalian subjects when administered orally or sublingually, to the extent that glyceryl trinitrate as of the time of the filing of this specification still enjoys widespread prescription by physicians for the treatment of angina pectoris.

It is also known that organic nitric esters are chemically unstable and may have a propensity towards decomposition with explosive violence. Nitric esters are especially unstable under acidic conditions, and since the preparation of nitric esters typically involves nitration of a hydroxy compound using a mixture of strong acid known in the art as “nitration acid”, the ester product of the reaction inherently contains trace quantities of acid residues for which care must be taken to remove the last traces of, to reduce the dangerous sensitivity to initiation. Thus, during the manufacturing process of say, glyceryl trinitrate, extra care is taken to remove the last traces of acidic residues from the product, for example by air agitation of the glyceryl trinitrate in the presence of aqueous bicarbonate for extended periods of time. However, even the most seemingly thorough treatment to remove acidic residues invariably almost always leaves some acid present in the nitration product. Nobel's Safety Powder™ was successful in part owing to the fact that it included an alkaline component whose function was to neutralize acidic residues. The peculiar property of the nitric esters to tenaciously retain acidic residues has been a long-standing problem in the art, and many accidents of the past have been attributed to the presence of acidic residues in nitric esters.

Since acidic residues are very difficult to completely remove from nitric esters even when contacting the nitric esters with alkaline aqueous solutions, it would be of benefit if there existed a material which is capable of neutralizing acidic residues, and which is also compatible with the nitric esters. It would be of further benefit if such a material also itself contained at least one nitric ester group, for cases in which explosive compositions having an increased degree of stability over the prior art were desired, which stabilized explosive compositions employ an acidity control agent is itself not inert, but which also contributes to the explosiveness of the composition. Some of the materials provided by the present invention have these properties.

In addition, it would be of benefit to provide the medicinal arts with new nitric esters having vasodilatory properties, for use either alone or co-administered with other medicinal compounds in the treatment of various cardio-vascular ailments. The present invention provides such materials.

### SUMMARY OF THE INVENTION

The present invention provides compounds according to the general formula: 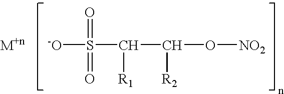
in which the cation M is selected from the group consisting of: hydrogen, alkali metal cations, alkaline earth metal cations, Group III metal cations, transition metal cations, ammonium ions (including organic-substituted ammonium cations), and including mixtures of any two or more of the foregoing; R₁ and R₂ are each independently selected from the group consisting of: hydrogen, methyl, and ethyl; and in which n represents the valence of the cation, including without limitation, the integers 1, 2, and 3.

The invention also provides materials of the general formula: O₂NO(CH₂)ₙSO₃H in which n is any integer between about 2 and about 24, and in general provides materials having one nitric ester unit and one sulfonate (or sulfonic acid) group in the same molecule, wherein the nitric ester unit and the sulfonate (or sulfonic acid) group are separated by at least one carbon atom.

The present invention also provides buffers, which include the aforedescribed materials in combination with one or more pairs of conjugate acid/base pairs known in the art to be useful as buffers. The present invention includes buffers in which one chemical species of the buffer includes an anionic form of a nitric ester provided by the present invention.

### DETAILED DESCRIPTION

The present invention provides compositions of matter heretofore undescribed in the chemical literature. Specifically, the invention provides compounds according to the general formula: 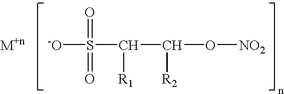
in which the cation M is selected from the group consisting of: hydrogen, alkali metal cations, alkaline earth metal cations, Group III metal cations, transition metal cations, ammonium ions (including organic-substituted ammonium cations), and including mixtures of any two or more of the foregoing; R₁ and R₂ are each independently selected from the group consisting of: hydrogen, methyl, and ethyl; and in which n represents the valence of the cation, including the integers 1, 2, and 3. As used in this specification and the claims appended hereto, “transition metal cations” includes not only simple cations of transition metals such as Au³⁺, Zn²+, Ag¹⁺, but also complex cations which contain a transition metal such as those described in “Advanced Inorganic Chemistry” fifth edition, by Cotton and Wilkinson (1988), pp 908-912, and similar and analogous known complex cations involving other metals.

The novel isethionyl nitrates of the present invention may be prepared by the direct nitration of isethionic acid using nitration acid, which nitration acid is produced by mixing two volumes of concentrated sulfuric acid, preferably containing at least 98% H₂SO₄ by weight, with one volume of concentrated nitric acid, preferably containing at least 70% by weight of HNO₃.

Isethionic acid may be prepared by first preparing an isethionate salt by reacting an alkylene oxide, including but not limited to, ethylene oxide, propylene oxide, and butylene oxide, with an aqueous solution of sodium bisulfite, as has been quite well known in the art for over 40 years. The salt so produced is then treated with acid, such as hydrochloric acid according to U.S. Pat. No. 4,499,028 to yield isethionic acid. The corresponding acids of the reaction products of propylene oxide, butylenes oxide, etc. are prepared in analogous fashion. Alternatively, isethionic acid is available commercially from a variety of suppliers, including Hubei Huanggang Yongan Pharmaceutical Co., Ltd. No. 44 Tuanhuang Road, Tuanfeng, Hubei, China. The word “isethionate” is used in a general sense herein, and the materials made from propylene oxide or butylene oxide reacted with aqueous bisulfite are considered to be substituted isethionates.

### EXAMPLE 1

The reader is cautioned that this procedure produces a material which is a powerful coronary vasodilator, which if uncontrollably admitted into the human body may cause increased heart rate, dizziness, headache, hallucinations, and even death. Since the properties of the compounds provided herein have not been fully investigated, it must be assumed a priori that they possess unknown effects on the human body and organs. In addition, nitric esters may spontaneously decompose, sometimes unprovoked, with explosive violence. Due care known only to those particularly skilled in the art of nitration must therefore be exercised when working with materials of this invention, to avoid catastrophe.

At room temperature, thirty milliliters of concentrated nitric acid containing 70% by weight of HNO₃ is poured into a 250 ml beaker, and to this nitric acid is slowly added sixty milliliters of concentrated sulfuric acid containing 98% by weight of H₂SO₄, with stirring. The temperature of the contents of the beaker rise to about 60 degrees centigrade, after which time the beaker containing the mixed acid is placed into an ice bath, until the temperature of the mixed acid has cooled to about 5 degrees centigrade. Subsequently, five milliliters of isethionic acid of about 98% purity is added dropwise to the mixed acid, with stirring, at such a rate that the temperature of the mixed acid is not permitted to rise above about 10 degrees centigrade. The contents of the beaker are permitted to rest for 15 minutes while being kept below about 10 degrees centigrade, after which time the contents of the beaker are poured slowly over about 300 grams of cracked ice contained in a 500 ml beaker. When the mixed acids contact the cracked ice, heat is generated, which melts a substantial portion of the ice. The remaining ice is allowed to melt, providing a total liquid volume of about 400 milliliters within the 500 ml beaker. At this stage, the liquid contains sulfuric acid, nitric acid, and the crude isethionyl nitrate product, which is an acid that corresponds to the formula 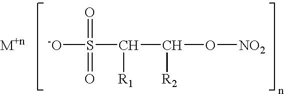
in which M⁺, R₁, and R₂ are each hydrogen, and n=1, thus affording the compound having formula C₂H₅SNO₆ having a formula weight of about 171.14.

The aqueous solution containing the mixed acids was transferred to a 1000 ml separatory funnel and extracted with 3 thirty milliliter aliquots of ethyl ether, which yielded a pale yellow solution. The ether extracts were combined, dried (MgSO₄), and evaporated to yield a slightly brown oily residue containing a high purity material which when diluted in approximately 100 times its volume of 190 proof grain alcohol was acidic to the taste, and which promptly caused the same symptoms as a small amount of glyceryl trinitrate (“NG”) when placed under my tongue. However, the effect of this novel substance seemed to be longer-lived than in my experiences with glyceryl trinitrate, which is surprising, since each molecule of the present substances contain only one nitric ester unit per molecule, compared with the three nitric ester units in the NG molecule. I therefore surmise that the remainder of the molecule of the instant substances somehow hinder their metabolism by the human organism, and are therefore able to exert a longer-lasting effect, which in my experience was on the order of about 1 hour. In the case of NG, a similar effect is only observed for about 7-8 minutes in me personally, and possibly up to a maximum of about 15 minutes. Therefore, the compounds of this invention are anticipated to be of great therapeutic value, owing not only to their apparent increased potency over NG, but also owing to their improved stability as described below.

A quantum leap increase in stability becomes possible because one may dissolve the acid forms of the materials in dry ether or other suitable solvent, and treat the solution so formed with any one of many possible metallic carbonates (or other metal salts) to yield the corresponding metallic salts of isethionyl nitrate esters. In such fashion, the alkali metal salts, alkaline earth metal salts, and transition metal salts of these materials may be prepared.

### EXAMPLE 2

Example 1 was repeated three times to afford a total combined volume of the acid form of isethionyl nitrate, which I have termed isethionic nitrate, abbreviated “IN”, of about 10 milliliters. I attempted to detonate this oil by placing one drop of it on an anvil and striking with a hammer, but was unable to obtain a detonation after many attempts. An equal volume of NG readily explodes with violence when similarly subjected. Thus, I surmise the compositions of the invention, in addition to being of greater therapeutic effect than NG, are also inherently more stable than NG. I recall that Nobel had originally been able to stabilize NG by neutralizing the last traces of acid residues present in it by addition of kieselguhr, Fuller's earth, diatomaceous earth, etc. The instant compositions are rendered even more stable by their being able to be stoichiometrically neutralized via chemical reaction with alkaline salts such as carbonates. One milliliter of IN (c.a. 0.006 moles) was dissolved in 30 ml of dry ether, to which was added 0.59 grams of NaHCO₃ (0.007 moles) with manual stirring with a glass rod. Effervescence was observed, indicating the neutralization had occurred. After evaporating the ether and drying, a slightly brownish white salt was obtained which corresponds to the formula above in which M=Na, n=1, and R₁ and R₂ are each hydrogen and corresponding to Na (C₂H₄NO₆).

### EXAMPLE 3

The procedure set forth in example 2 was repeated, suspending about 1 ml of IN in dry ether and adding 0.007 moles of CaCO₃ with the same processing to obtain the calcium salt of IN, corresponding to Ca (C₂H₄NO₆)₂.

### EXAMPLE 4

The procedure set forth in example 2 was repeated, suspending about 1 ml of IN in dry ether and adding 0.007 moles of MgCO₃ with the same processing to obtain the magnesium salt of IN, corresponding to Mg (C₂H₄NO₆)₂.

### EXAMPLE 5

The procedure set forth in example 2 was repeated, suspending about 1 ml of IN in dry ether and adding 0.007 moles of KHCO₃ with the same processing to obtain the potassium salt of IN, corresponding to K(C₂H₄NO₆).

### EXAMPLE 6

The procedure set forth in example 2 was repeated, suspending about 1 ml of IN in dry ether and adding 0.007 moles of ZnCO₃ with the same processing to obtain the zinc salt of IN, corresponding to Zn (C₂H₄NO₆)₂.

Thus, from the foregoing, one of ordinary skill immediately recognizes that the present invention enables one of ordinary skill to prepare any metal salt of IN, by combining the carbonate or bicarbonate salt of the desired metal with IN in a suitable solvent.

Similarly, other salts are preparable by addition of the corresponding basic substance to the IN. For example, the same molar quantity of a material selected from: ammonia, a mono-alkyl amine, a di-alkyl amine, and a tri-alkyl amine may be substituted for the metal carbonate in the foregoing examples to yield the corresponding ammonium salt. Such materials may be added as a solution in an organic solvent, water, or may, in the cases of volatile amines, be bubbled (sparged) into a solution of an IN.

Further, complex cations may be incorporated into a compound that comprises IN by first forming a solution containing the complex cation, (preferably a hydroxide compound, but inorganic salts are also deemed suitable, such as halides, sulfates, phosphates, etc.) and mixing the solution with the solution of IN, which may be either a solution containing an organic solvent or aqueous solution.

While diethyl ether was used in the foregoing examples, other solvents are useful herein, including without limitation C₁-C₁₀ alcohols, alkyl ethers, aromatics such as benzene, toluene, xylene, etc., glycol ethers, etc. Diethyl ether is especially preferred since it is well accepted for use in preparing pharmaceutical compositions of matter, and is readily volatilized for ease in its removal.

An alternate method for preparing IN is to add 50 milliliters of strong (70%) nitric acid to a 100 ml beaker housed in an ice bath, and to add 10 grams of phosphoric oxide P₂O₅) to the nitric acid slowly in about 1 gram quantities, allowing for cooling and with stirring. The contents of the beaker are then chilled to 5 degrees centigrade and 7 ml of isethionic acid are added slowly with stirring while maintaining the beaker contents below about 10 degrees centigrade. Extraction with ether and worked up as described earlier affords IN.

Other cations corresponding to M in the above formulae than metals are also possible for materials of the present invention, such as ammonium cations, and cations of mono-, di-, and even tri-substituted amino compounds. These materials are readily prepared by reacting the corresponding ammonium hydroxide, alkylammonium hydroxide, dialkylammonium hydroxide or trialkylammonium hydroxide, with the solution of IN in ether, evaporating the ether, and drying. By altering the size of and number of substituents on the ammonium cations, it is possible to provide a wide range of vasodilator compounds having different absorption rates into the human organism, and of varying durations of effect due to the nature of the substituents on the amino nitrogen. The present invention also contemplates situations where the sulfuric acid residue of the IN molecule is esterified, i.e., wherein the M in the above formulae is a hydrocarbyl moiety, including without limitation alkyl moieties of any chain length in the range of about C₁ to C₂₄ by treatment of the isethionic acid with an alkylating agent, such as diethyl sulfate, and subjecting the resulting ester to nitration as hereinbefore described to afford the mixed alkyl-nitric ester of isethionic acid, which is anticipated to have similar medicinal properties. However, since many sulfuric esters are toxic, this embodiment is believed to be limited to the ethyl esters, and is less preferable owing to the absence of the ionic —SO₃ group.

In a general sense, one form of the present invention provides nitric esters corresponding to the general formula:
[O₂NO—R—SO₃]ₙM⁺ⁿ
in which M is selected from the group consisting of: hydrogen, alkali metal cations, alkaline earth cations, Group III metal cations, transition metal cations, ammonium cation, mono-alkyl substituted ammonium, di-alkyl substituted ammonium, tri-alkyl substituted ammonium, n is any integer which corresponds to the valency of the M, and in which R is any C₂ to C₂₄ hydrocarbyl radical.

### Buffers

One advantage of the materials provided by the present invention over compounds of the prior art used in treating angina is that the instant materials may be used in combination with a buffer. In one embodiment of the invention, the instant materials are used themselves as one of the salts in a buffer pair. The exact buffer chosen is not critical as long as a desired pH in the range of between about 5 and about 9 is maintained. Any buffer known to those skilled in the art may be used in combination with a material of the present for this purpose. Suitable buffer systems thus include without limitation: phosphate buffers; sulfate buffers; acetic/acetate buffers; C₁-C₁₀ mono- and polycarboxylic acid buffers; substituted carboxylic acids such as lactic, ascorbic, and tartaric acid buffers; and carboxylic acids that have unsaturation such as maleic and furmaric buffers. The present invention contemplates use of all buffer systems known to those skilled in the art. Buffer systems are known to contain salt pairs. Currently, the most preferred buffer is an alkali or alkaline earth dihydrogen phosphate, adjusted to a pH of about 7.3.

To provide a buffer according to one preferred form of the invention, one first provides an aqueous solution containing 0.1 moles (about 17 grams) of IN dissolved in sufficient water to bring the total volume to about 50 milliliters. Next, 0.1 moles of Ca(OH)₂ is added (calcium carbonate may also be used) to thus provide a solution containing 0.15 moles of a material having the formula: Ca(OH)(O₃SCH₂CH₂ONO₂). In this compound, of the two hydroxy groups initially present on the calcium hydroxide, only one has been replaced by an isethionyl nitrate anion owing to the addition of that amount of IN which is sufficient to neutralize a single hydroxide on the calcium. An aqueous solution containing this salt of IN inherently contains residual alkalinity. The remaining hydroxy group on the calcium may be reacted, in a preferred form of the invention, via the slow addition of phosphoric acid to such a solution, to ultimately provide an aqueous solution of a salt having the empirical formula Ca(OH₂PO₃)(O₃SCH₂CH₂ONO₂). Of course, as is known to those skilled in this art, such an aqueous solution is in reality a mixture of various ionic species in dynamic equilibrium. However, the above mixed calcium salt of phosphoric acid and isethionyl nitrate is isolatable by drying the aqueous solution. In this example it is preferred to add sufficient phosphoric acid until a pH in the range of between about 6 and 9 is achieved, with a neutral to slightly alkaline or slightly acidic pH being most preferred. Currently, the most preferred pH is about 7.1-7.2, but any desired pH below about 10 may be achieved by addition of suitable acid.

Although an aqueous solution containing 0.1 mole of IN is suitable as described above for providing salts which contain an anionic form of IN useful in a buffer pair, the neutralization reaction may be conducted in an alcohol or any other suitable organic solvent, including glyme, diglyme, ethers, glycols, etc. In addition, various concentrations of IN may be employed, as no indications have been given thus far that the concentrations at which IN is useful in its reactions are limited. The only limitation which I have been able to discern is that IN begins to discolor when heated to about 70 degrees C. so it is desirable to avoid heating the material above 50 degrees C. during processing, including evaporating solvents in which it is dissolved. Otherwise, neutralization of IN is as straightforward as for other acids.

In similar fashion, it is possible to prepare analogous mixed salts using various divalent metals as the basis, including without limitation: the metal ions: magnesium, strontium, barium, aluminum, zinc, or any polyvalent metal cation, in the place of calcium. Although phosphoric acid was used in the foregoing example to neutralize the remaining hydroxy group on the calcium, any other mono-protic or poly-protic acid is suitable, including without limitation sulfuric acid, sulfurous acid, phosphoric acid, phosphorous acid, nitric acid, hydrochloric acid, hydrobromic acid, hydrofluoric acid, hydriodic acid, iodic acid, bromic acid, bromous acid, iodous acid, chlorous acid, hypoiodous acid, hypobromous acid, hypochlorous acid, osmic acid, chloroplatinic acid, rhodic acid, divalent and trivalent organic acids. Suitable examples of monocarboxylic acids containing one carboxy group include formic acid, acetic acid, propionic acid, butyric acid, valeric acid, caproic acid, enanthic acid, caprylic acid, pelargonic acid, capric acid, lauric acid, myristic acid, palmitic acid, stearic acid, and mixtures thereof. Still other suitable monocarboxylic acids are the monocarboxylic acids substituted by any of the following groups: CH3—(CH2)n, wherein n is an integer of value of at least 1, CH3, OH, NH2, Cl, Br, F, I, OR″, NHR″, NR″₂, NO₂, SO₃, cyclic rings like cyclopentane, cyclohexane, phenyl, benzyl, or a mixture of these substituents; wherein R″ is selected from saturated or unsaturated alkyl chains. Preferred examples are 1-methylcyclohexanecarboxylic acid, glycolic acid, mandelic acid, lactic acid, salicylic acid, benzoic acid, and derivatives thereof. The substituents may also be anywhere in the alkyl chain attached to the acidic function. The alkyl chain can be saturated or non saturated.

Still other typical of organic acids suitable for use herein as acid carrier includes the polycarboxylic acids containing two carboxy groups. Typical of these ingredients are selected from succinic acid, matonic acid, (ethylenedioxy) diacetic acid, maleic acid, diglycolic acid, tartaric acid, tartronic acid, fumaric acid, oxalic acid, glutaric acid, adipic acid, pimelic acid, suberic acid, azelaic acid, or sebacic acid, as well as the ether carboxylic acid and the sulfinyl carboxylic acids, and mixtures thereof.

Other dicarboxylic adds suitable for use herein in forming salts including an isethionyl nitrate are the dicarboxylic acids substituted by CH₃—(CH₂)ₙ, wherein n is an integer of value of at least 1, CH₃, OH, NH₂, Cl, Br, F, I, OR″, NHR″, NR″₂, NO₂, SO₃, cyclic rings like cyclopentane, cyclohexane, phenyl, benzyl, or a mixture of these substituents; wherein R″ is selected from saturated or unsaturated alkyl chain. Preferred examples of such substituted dicarboxylic acids are phtalic acid, isophtalic acid, terephtalic acid, malic acid, fumaric acid, tartaric acid, or mixtures thereof. The substituents may also be anywhere in the alkyl chain attached to the acidic functions. The alkyl chains can be saturated or non saturated.

Other polycarboxylic acids suitable are the polycarboxylic acids containing three carboxy groups and include, in particular, water-soluble citric acid, aconitric and citraconic acid as well as succinic derivatives such as the carboxymethyloxysuccinic described in British Patent No. 1,379,241, lactoxysuccinic described in British Patent No. 1,389,732, and aminosuccinic described in Netherlands Application 7205873, and the oxypolycarboxylic materials such as 2-oxa-1,1,3-propane tricarboxylic described in British Patent No. 1,387,447. Other suitable polycarboxylic acids are the polycarboxylic acids containing four carboxy groups and include oxydisuccinic disclosed in British Patent No. 1,261,829, 1,1,2,2-ethane tetracarboxylic, 1,1,3,3-propane tetracarboxylic and 1,1,2,3-propane tetracarboxylic. Polycarboxylic containing sulfo substituents include the sulfosuccinic derivatives disclosed in British Patent Nos. 1,398,421 and 1,398,422 and in U.S. Pat. No. 3,936,448, and the sulfonated pyrolysed citratic described in British Patent No. 1,439,000.

Alicyclic and heterocyclic polycarboxylic include cyclopentane-cis,cis,cis-tetracarboxylic, cyclopentadienide pentacarboxylic, 2,3,4,5-tetrahydrofuran-cis, cis, cis-tetracarboxylic, 2,5-tetrahydrofuran-cis-dicarboxylic, 2,2,5-tetrahydrofuran-tetracarboxylic, 1,2,3,4,5,6-hexane-hexacarboxylic, polyacrylic acid, polymaleic acid, polymaleic-acrylic acids, sugar-acids like glucose-phosphonic acid, gluconic acid, glucuronic acid, mannanic acid, galactonic acid, arabinamic acid, and carboxymethyl derivatives of polyhydric alcohols such as sorbitol, mannitol and xylitol. Aromatic polycarboxylic include mellitic acid, pyromellitic acid and the phthalic acid derivatives disclosed in British Patent No. 1,425,343.

Thus, in a general sense, one embodiment of the present invention provides nitric ester compounds of the formula: 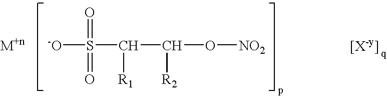
in which the cation M is selected from the group consisting of: hydrogen, alkali metal cations, alkaline earth metal cations, Group III metal cations, transition metal cations, ammonium ions (including organic-substituted ammonium cations), and including mixtures of any two or more of the foregoing; R₁ and R₂ are each independently selected from the group consisting of: hydrogen, a C₁ to C₆ hydrocarbyl group; n represents the valence of the cation, including the integers 1, 2, and 3; X is an anion or anionic species selected from the group consisting of: fluoride, chloride, bromide, iodide, nitrate, nitrite, phosphate, phosphate, monohydrogen phosphate, dihydrogen phosphate, monohydrogen sulfate, sulfate, carbonate, bicarbonate, hydroxide, carboxylate, dicarboxylate and polycarboxylate anions; y is the valence of the anionic species X and is generally 1, 2, or 3; p may be any integer including 1, 2, 3 etc., but p is not greater than n; q may be any integer selected from the group consisting of: 0, 1, 2, 3, etc., but q is not greater than n−p, subject to the proviso that the sum of p plus the product of (y times q) equals the valence n of the cation M and wherein p is not equal to zero.

For example, in the above structure, when M is calcium and p is 1, R₁ and R₂ are both hydrogen, X may be hydroxide, and both q and y are 1. Alternatively, M may be aluminum or rhodium (III), p may be 1, R₁ and R₂ are both hydrogen, X may be monohydrogen phosphate, in which case y=2 and q=1. Alternatively, M may be aluminum or rhodium (III), p may be 1, R₁ and R₂ are both hydrogen, X may be dihydrogen phosphate, in which case y=1 and q=2. By substituting any known anion for X, including without limitation those previously mentioned in this specification, the present invention provides a wide range of novel compounds. M may be any trivalent metal, R₁ and R₂ may both be hydrogen or a C₁ to C₆ hydrocarbyl group, p may be 1 or 2, q may be 0, 1, 2, or 3, and y may be 1, or 2. When M is a transition metal, including without limitation rhodium, gold, nickel, platinum, these compounds are believed to be useful as hardening additives or brightening additives when added to conventional electroplating baths in the same molar amount based on the isethionate portion of the molecule as isethionate compounds are currently employed in this same art.

Preparation of the wide range of compounds described in the two preceding paragraphs begins with IN, dissolved in a solvent such as an alcohol or water at a temperature of about 10-20 degrees C. To this solution may be added a compound which contains a metal atom M and a cation which is capable of accepting a proton from the IN. For example, one mole of an aqueous solution of Rh(PO₄) may be combined with one mole of aqueous IN to yield a solution which contains one mole of Rh(O₃SCH₂CH₂ONO₂)(HPO₄). By evaporating the water or other solvent in vacuo with a sweep of a stream of warm nitrogen, such a salt is believed to be isolatable. One of ordinary skill immediately recognizes that by simple ionic displacement a wide range of compounds are made possible by the present invention.

A compound of the present invention may also be part of a formulation which includes a buffer comprising a salt pair of which neither of the salts include an isethionyl nitrate species.

The materials of the present invention may also be administered to mammalian species transdermally. Various systems are known in the prior art for administering glyceryl trinitrate, and include the following patent documents, all of which are herein incorporated by reference thereto in their entirety: U.S. Pat. Nos. 5,817,697; 5,302,395; 5,262,165; 5,202,125; 4,751,087; 4,696,821; 4,681,584; 4,615,699; and 4,336,243, including patent documents and references cited in each. These prior art patents provide pharmaceutically-acceptable transdermal delivery vehicles suitable for use with the compounds of the present invention. In addition, transdermal delivery systems for delivering ionic compounds to mammalian subjects are well-known in the art.

Thus, the present invention also provides compounds which are suitable as ingredients in skin patches suitable for adhering to the skin and delivering, over time, transdermally, to a human subject a composition comprising one or more nitric ester compounds corresponding to the formula: 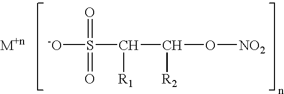
in which M is selected from the group consisting of: hydrogen, alkali metal cations, alkaline earth cations, ammonium cation, mono-alkyl substituted ammonium, di-alkyl substituted ammonium, tri-alkyl substituted ammonium, n is any integer which corresponds to the valency of the M, and R₁ and R₂ are each independently selected from the group consisting of hydrogen, methyl, and ethyl. The new nitric esters of this invention are useful with all such systems which contain no ingredients that cause instability in nitric esters. However, as mentioned, the ability to use alkaline metals such as calcium, barium, strontium, or magnesium ions (including combinations of the foregoing) as M in the above formula renders great control over the pH of the composition. One principle would be illustrated by mixing 2.0 moles of Ca(OH)₂ with less than 2.0 moles of the acid form of isethionyl nitrate, say 1.9 moles of isethionyl nitrate, to yield an isolatable calcium salt of isethionyl nitrate having excess alkalinity which would prove to provide an extremely time-stable nitric ester administrable to human subjects having a heretofore unseen shelf-life. One of ordinary skill in the art immediately recognizes that any amount of excess alkalinity as described above in an IN salt, especially when polyvalent metal cations are present, is within the scope these teachings.

Various workers in the prior art have provided compositions and methods useful for administering NG to patients, which are described in one or more of U.S. Pat. Nos. 6,808,514; 6,773,716; 6,706,732; 6,706,689; 6,673,802; 6,660,756; 6,635,273; 6,632,460; 6,627,234; 6,586,478; 6,562,838; 6,559,180; 6,548,490; 6,541,487; 6,537,992; 6,476,021; 6,475,534; 6,462,047; 6,458,804; 6,451,813; 6,433,019; 6,426,084; 6,403,597; 6,284,790; 6,264,981; 6,258,373; 6,214,379; 6,190,691; 6,187,790; 6,187,314; 6,166,061; 6,143,757; 6,143,746; 6,140,319; 6,132,757; 6,132,753; 6,100,286; 6,051,594; 6,043,252; 6,030,621; 5,981,563; 5,973,011; 5,962,477; 5,945,123; 5,855,908; 5,811,416; 5,785,989; 5,767,160; 5,744,124; 5,731,339; 5,698,738; 5,665,766; 5,565,466; 5,543,430; 5,489,610; 5,484,602; 5,288,498; 5,288,497; 5,278,192; 5,252,600; 5,236,713; 5,192,799; 5,187,305; 5,175,187; 5,155,120; 5,132,114; 5,122,127; 5,087,631; 5,047,230; 5,001,151; 4,921,695; 4,885,173; 4,879,308; 4,863,737; 4,846,826; 4,842,854; 4,834,985; 4,828,836; 4,764,378; 4,713,239; 4,704,119; 4,696,821; 4,659,717; 4,650,484; 4,533,540; 4,486,193; 4,482,534; 4,465,191; 4,428,925; 4,421,737; 4,389,393; 4,369,172; 4,357,469; 4,323,577; 4,226,849; 4,200,640; and 4,091,091, all of which are herein fully incorporated by reference thereto, including all references cited in each patent. These methods are useful for administering the inventive IN of this invention and its salts by substituting IN in the stead of NG in these compositions, on a weight-for-weight basis, or on a molar basis based on the molar quantity of total nitric ester present.

It is further within the scope of this invention to employ any hydroxy-terminated alkyl sulfate as a raw material for nitration to produce a nitric ester useful in treating the symptoms of angina pectoris. This includes all materials of the formula: HO(CH₂)ₙSO₃H in which n is any integer between about 2 and about 24, which are preparable by reacting the corresponding alkylene oxide with an aqueous solution of bisulfite ions, followed by acidification and extraction as hereinbefore described. The nitric esters of these materials may be utilized as herein described to yield nitric esters having the uses taught herein, corresponding to the general formula: O₂NO(CH₂)ₙSO₃H in which n is any integer between about 2 and about 24. The invention also contemplates such materials in which the alkyl chain is branched or comprises a cyclic hydrocarbyl moiety. “Hydrocarbyl”, as used in this specification and the appended claims when referring to a substituent or group is used in its ordinary sense, which is well-known to those skilled in the art. Specifically, it means a group having a carbon atom directly attached to the remainder of the molecule and having predominantly hydrocarbon character. Examples of hydrocarbyl substituents or groups include: 1) hydrocarbon substituents, that is, aliphatic (e.g., alkyl or alkenyl), alicyclic (e.g., cycloalkyl, cycloalkenyl) substituents, and aromatic-, aliphatic-, and alicyclic-substituted aromatic substituents, as well as cyclic substituents wherein the ring is completed through another portion of the molecule (e.g., two substituents together form an alicyclic radical); 2) substituted hydrocarbon substituents, that is, substituents containing non-hydrocarbon groups which, in the context of this invention, do not alter the predominantly hydrocarbon substituent (e.g., halo (especially chloro and fluoro), hydroxy, alkoxy, mercapto, alkylmercapto, nitro, nitroso, and sulfoxy); 3) hetero substituents, that is, substituents which, while having a predominantly hydrocarbon character, in the context of this invention, contain other than carbon in a ring or chain otherwise composed of carbon atoms. Heteroatoms include sulfur, oxygen, nitrogen, and encompass substituents as pyridyl, furyl, thienyl and imidazolyl. In general, no more than two, preferably no more than one, non-hydrocarbon substituent will be present for every ten carbon atoms in the hydrocarbyl group; typically, there will be no non-hydrocarbon substituents in the hydrocarbyl group.

By evaporating the solvent from the solutions in which various ionic species as herein described are prepared, it is possible to provide a wide range of dry, stable salts of IN. These solid salts may be compounded and blended with other pharmaceutically-acceptable materials, as are known in the art.

Finished medicinal compositions according to the invention may be prepared by conventional mixing or co-comminuting the IN salt component and other known pharmaceutically-acceptable components with one another, as such methods of mixing medicinal substances with one another are known in the art. According to another form of the invention, a suspension in oil of the respective components may be prepared and mixed. According to one preferred form of the invention, an oil suspension containing the components of a composition according to the invention may be contained in a softgel capsule or other capsule, as such preparations are known to those in the pharmaceutical arts. In cases where it is desired to provide a combination according to the invention in the form of a tablet or pill, various binders, carriers, and excipients, the use of which are well-known to those skilled in the art, may be included in the inventive combinations.

Another use for the isethionyl nitrates provided by this invention, including the acid forms of the nitric esters described in this specification, and especially the salts as described above which contain non-neutralized hydroxy groups or otherwise comprise inherent residual alkalinity, as a moderator or stabilizer for explosive compositions which include other nitric esters, such as, but not limited to, NG, pentaerythritol tetranitrate, tetryl, trinitrotoluene, dinitrotoluenes, picric acid and picrates, HMX, cyclonite, nitric esters of glycols such as EGDN, Semtex, dynamites, nitric esters of starches, guanidine nitrate, and all known explosive compositions comprising a nitric ester, since they all suffer from the same inherent causes of instability. Having present in such compositions a nitric ester which itself is alkaline in nature adds to the stability of such compositions, by the inclusion of an alkaline material (the IN salts from above containing inherent alkalinity) which is highly compatible and miscible with the main explosive ingredient itself. Thus, the present invention includes substitution of stabilizers of the prior art in explosive compositions which comprise a nitric ester with a salt of a nitric ester provided by the present invention.

One non-limiting example of this would be a mixture comprising glyceryl trinitrate, cyclonite, pentaerythritol tetranitrate, or cyclotetramethylene-tetranitramine with between about 0.01% and about 2% by weight of one or more nitric ester compounds corresponding to the general formula:
[O₂NO—R—SO₃]ₙM⁺ⁿ
in which M is selected from the group consisting of: hydrogen, alkali metal cations, alkaline earth cations, Group III metal cations, transition metal cations, ammonium cation, mono-alkyl substituted ammonium cations, di-alkyl substituted ammonium cations, tri-alkyl substituted ammonium cations, n is an integer which corresponds to the valency of the cation M, and R is any C₂ to C₂₄ hydrocarbyl radical. All other compounds described herein should be useful in this regard as well.

The nitric ester compounds of the invention may be administered alone or in combination with pharmaceutically acceptable carriers or diluents, preferably by sublingual administration, but also possible by other known routes of administration of nitric esters. Such administration may be carried out in single or multiple doses. More particularly, the active compounds may be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored. In general, the active compounds are present in such dosage forms at concentration levels ranging from about 0.001% to about 70% by weight.

For oral administration of a composition according to the invention, tablets containing various excipients such as microcrystalline cellulose, sodium citrate, calcium carbonate, dicalcium phosphate and glycine may be employed along with various disintegrants or fillers such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. These materials are well known in the art as binders, excipients and fillers, but the present invention is not limited to the use of such materials in the medicaments it provides, it being possible to employ all known binders, excipients, and fillers, etc. in combination with the nitric ester compounds provided herein. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active compound may be combined with various sweetening or flavoring agents, coloring matter or dyes, and, if so desired, emulsifying and/or suspending agents as well, together with such diluents as water, ethanol, propylene glycol, glycerin and various like combinations thereof.

The nitric ester compounds of the present invention are believed to be especially safe to administer, it being submitted that the nitric esters of the invention in ordinary metabolism are broken down through de-esterification to the isethionate ion. Isethionate ion has been observed itself to possess some vasodilating activity. [Sasajima, et al.; “Effects of Taurine and Isethionate on the Lactic acid-induced Inhibition of Rat Gastrocnemius Muscle Contraction” Oyo Yakuri 8(6) 697 (1974)]. Thus, I propose that the long-lasting duration of the vasodilatory effect of the IN which I observed in myself relative to NG is most likely due to a synergistic effect from the simultaneous effects of IN and the isethionate ion, its metabolite on the human organism. Therefore, a composition according to the invention when used to treat angina pectoris exhibits a dual-acting synergy, which is a feature neither taught nor suggested in compounds or literature of the prior art indicated for such treatment.

The Japanese use taurine (chemically 2-amino ethanesulfonic acid) therapy in the treatment of ischemic heart disease, because from a well-known study where, low taurine and magnesium levels were found in patients after heart attacks. Like magnesium, taurine affects cell membrane electrical excitability by normalizing potassium flow in and out of heart muscle cells. At times when the human organism is deficient in magnesium, for whatever reason, possibly owing to consumption of alcohol, or chronic tobacco use, it is known that the amino acid taurine, may act as a substitute for magnesium in the heart muscle in regulating potassium. I have firsthand knowledge of these effects, as I personally formerly would suffer from arrhythmias, typically after consuming caffeinated beverages. At times, I could measure my heart rate at a level of about 180 beats per minute, which was the cause of much mental anxiety. These arrhythmia spells would typically last 10-30 minutes. By self-administering 250 mg of taurine in conjunction with magnesium and calcium, I discovered that these arrhythmias could be alleviated wholly, and by supplementing my diet with one 250 mg tablet of taurine on a weekly basis, I have not suffered such arrhythmia for several years. Further, a large-scale study in Japan drawing from 24 populations in 16 countries revealed a strong, inverse association between levels of taurine excretion and ischemic heart disease. [Hypertens Res 2001 July;24(4): pp. 453-7].

The structural similarity between the IN I have prepared for the first time ever and the amino acid taurine is no accident. By my design, I have herein provided an effective material for treating heart conditions whose molecular structure “fits” within the receptor sites and is metabolically compatible therewith. Thus, I propose that the compounds of the present invention be co-administered with taurine. In fact, the taurate anion is ideally suited to function as the anion “X” in the relevant structures in foregoing specification and the appended claims. Mixing aqueous solutions which each contain an equimolar amount of taurine and the nitric ester of isethionic acid (IN) with one another yields isethionyl nitrate taurate [O₂NOCH₂CH₂SO₃⁻, ^{+H}₃NCH₂CH₂SO₃H] (empirical formula C₄H₁₂S₂N₂O₉), which is probably better referred to by the proposed common name of taurinium isethionyl nitrate, or simply, “TIN”.

The present invention further provides taurine in combination with all compositions claimed herein, wherein taurine is present in any amount of the composition between 0.01% and about 99%. Any salt of IN may be adsorbed onto taurine, as a carrier, by spraying IN neat, or spraying or otherwise combining IN with taurine and drying to remove water.

Consideration must be given to the fact that although this invention has been described and disclosed in relation to certain preferred embodiments, obvious equivalent modifications and alterations thereof will become apparent to one of ordinary skill in this art upon reading and understanding this specification and the claims appended hereto. This includes the subject matter defined by any combination of any one of the various claims appended hereto with any one or more of the remaining claims, including the incorporation of the features and/or limitations of any dependent claim, singly or in combination with features and/or limitations of any one or more of the other dependent claims, with features and/or limitations of any one or more of the independent claims, with the remaining dependent claims in their original text being read and applied to any independent claim so modified. This also includes combination of the features and/or limitations of one or more of the independent claims with the features and/or limitations of another independent claim to arrive at a modified independent claim, with the remaining dependent claims in their original text being read and applied to any independent claim so modified. Accordingly, the presently disclosed invention is intended to cover all such modifications and alterations, and is limited only by the scope of the claims which follow, in view of the contents of this specification.

What is claimed is:

1) A composition of matter which comprises at least one nitric ester compound corresponding to the formula: 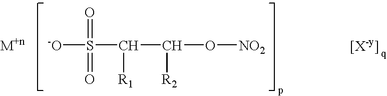
in which the cation M is selected from the group consisting of: hydrogen, alkali metal cations, alkaline earth metal cations, Group III metal cations, transition metal cations, ammonium ions (including organic-substituted ammonium cations), including mixtures of any two or more of the foregoing; R₁ and R₂ are each independently selected from the group consisting of: hydrogen and a C₁ to C₆ hydrocarbyl group; n represents the valence of the cation, including the integers 1, 2, and 3; X is an anion selected from the group consisting of: fluoride, chloride, bromide, nitrate, nitrite, phosphate, phosphate, monohydrogen phosphate, taurate, dihydrogen phosphate, monohydrogen sulfate, sulfate, carbonate, bicarbonate, hydroxide, carboxylate, dicarboxylate, and polycarboxylate anions having more than two carboxyl groups; y is the valence of the anionic species X and may be 1, 2, or 3; p may be any integer including 1, 2, and 3, subject to the proviso that p is not greater than n; q may be any integer selected from the group consisting of: 0, 1, 2, 3 subject to the proviso that q is not greater than n−p, and further subject to the provisos that the sum of p plus the product of (y times q) equals the valence n of the cation M and that p is not equal to zero.

2) A composition according to claim 1 wherein M is selected from the group consisting of: magnesium and calcium; X is selected from the group consisting of: hydroxide, bicarbonate, and taurate; and wherein p=1; q=1, y=1 and n=2.

3) A composition according to claim 1 wherein M is selected from the group consisting of: magnesium and calcium; X is taurate; and wherein p=1; q=1, y=1 and n=2.

4) A composition according to claim 1 wherein M is selected from the group consisting of: magnesium and calcium; X is hydroxide; and wherein p=1; q=1, y=1 and n=2.

5) A composition according to claim 1 wherein M is magnesium; X is taurate; p=1; q=1; y=1 and n=2.

6) A composition according to claim 1 wherein M is calcium; X is taurate; p=1; q=1; y=1;and n=2.

7) A salt which contains a cationic portion that comprises a polyvalent cation, and an anionic portion which comprises a first anion which comprises a nitric ester group and a second anion which comprises an alkaline residue.

8) A salt according to claim 7 in which the polyvalent cation is any alkaline earth metal.

9) A salt according to claim 7 in which said alkaline residue is selected from the group consisting of: hydroxide ion, carbonate ion, bicarbonate ion, sulfate ion, bisulfate ion, phosphate ion, monohydrogen phosphate ion, dihydrogen phosphate ion, nitrate ion, borate ion, hydrogen borate ion, dihydrogen borate ion, and oxide ion.

10) A composition according to claim 1 which further comprises a second, different nitric ester compound corresponding to the general formula. 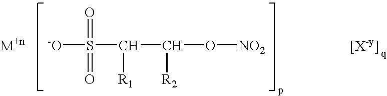
in which the cation M is selected from the group consisting of: hydrogen, alkali metal cations, alkaline earth metal cations, Group III metal cations, transition metal cations, ammonium ions (including organic-substituted ammonium cations), including mixtures of any two or more of the foregoing; R₁ and R₂ are each independently selected from the group consisting of: hydrogen and a C₁ to C₆ hydrocarbyl group; n represents the valence of the cation, including the integers 1, 2, and 3; X is an anion selected from the group consisting of: fluoride, chloride, bromide, iodide, nitrate, nitrite, phosphate, phosphate, monohydrogen phosphate, taurate, dihydrogen phosphate, monohydrogen sulfate, sulfate, carbonate, bicarbonate, hydroxide, carboxylate, dicarboxylate and polycarboxylate anions; y is the valence of the anionic species X and may be 1, 2, or 3; p may be any integer including 1, 2, and 3, subject to the proviso that p is not greater than n; q may be any integer selected from the group consisting of: 0, 1, 2, 3 subject to the proviso that q is not greater than n−p, and further subject to the provisos that the sum of p plus the product of (y times q) equals the valence n of the cation M and that p is not equal to zero.

11) A composition according to claim 10 wherein in one of the compounds present M is selected from the group consisting of: magnesium and calcium; and X is selected from the group consisting of: hydroxide, bicarbonate, and taurate; and wherein p=1; q=1, y=1 and n=2.

12) A buffer which comprises a composition according to claim 1 and which further comprises a material selected from the group consisting of: a conjugate acid or a conjugate base of a chemical species selected from the group consisting of: M, X, or the isethionyl species in the formula of claim 1.

13) A buffer according to claim 12 which, when dissolved in water provides a solution having a pH in the range of between about 5 and about 9.

14) A single-dose medicament comprising:
a) at least one nitric ester compound according to claim 1; and
b) a pharmaceutically acceptable material selected from the group consisting of: binders, carriers, and excipients.

15) A single dose medicament according to claim 14 in which the total amount of nitric ester compounds in said medicament is any amount in the range of between about 0.001 milligrams to about 60 milligrams.

16) A single dose medicament according to claim 15 in which M is selected from the group consisting of: magnesium and calcium; X is selected from the group consisting of: hydroxide, bicarbonate, and taurate; and wherein p=1; q=1, y=1 and n=2.

17) A skin patch suitable for being adhered to human skin and delivering, over time, transdermally, to a human subject, a composition which comprises:
a) at least one nitric ester compound corresponding to the formula: 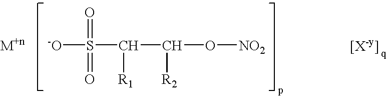
in which the cation M is selected from the group consisting of: hydrogen, alkali metal cations, alkaline earth metal cations, Group III metal cations, transition metal cations, ammonium ions (including organic-substituted ammonium cations), including mixtures of any two or more of the foregoing; R₁ and R₂ are each independently selected from the group consisting of:
hydrogen and a C₁ to C₆ hydrocarbyl group; n represents the valence of the cation, including the integers 1, 2, and 3; X is an anion selected from the group consisting of: fluoride, chloride, bromide, iodide, nitrate, nitrite, phosphate, phosphate, monohydrogen phosphate, taurate, dihydrogen phosphate, monohydrogen sulfate, sulfate, carbonate, bicarbonate, hydroxide, carboxylate, dicarboxylate and polycarboxylate anions; y is the valence of the anionic species X and may be 1, 2, or 3; p may be any integer including 1, 2, and 3, subject to the proviso that p is not greater than n; q may be any integer selected from the group consisting of: 0, 1, 2, 3 subject to the proviso that q is not greater than n−p, and further subject to the provisos that the sum of p plus the product of (y times q) equals the valence n of the cation M and that p is not equal to zero; and
b) a pharmaceutically-acceptable transdermal delivery vehicle.

18) A skin patch according to claim 17 wherein M is selected from the group consisting of: magnesium and calcium; X is selected from the group consisting of: hydroxide, bicarbonate, and taurate; and wherein p=1; q=1, y=1 and n=2.
